(19) United States
(12) Patent Application Publication
(10) Pub. No.: US 2026/0053559 A1
(43) Pub. Date: Feb. 26, 2026

(54) CONFIGURABLE MAP AND ABLATE CATHETER

(71) Applicant: Boston Scientific Scimed, Inc., Maple Grove, MN (US)

(72) Inventor: Michael Sean Coe, Plymouth, MN (US)

(21) Appl. No.: 19/306,876

(22) Filed: Aug. 21, 2025

Related U.S. Application Data
(60) Provisional application No. 63/686,013, filed on Aug. 22, 2024.

(51) Int. Cl. A61B 18/14; A61B 18/00

(52) U.S. Cl. CPC A61B 18/1492; A61B 2018/00267; A61B 2018/00351; A61B 2018/00577; A61B 2018/00613; A61B 2018/00642; A61B 2018/00839; A61B 2018/1467

(57) ABSTRACT

A catheter to detect physiological signals from within the patient's heart is disclosed. The catheter includes electrode assemblies extending distally from a distal region of a shaft and coupled together at a distal hub. A first electrode assembly of the electrode assemblies is transitionable between a first expanded configuration and a first collapsed configuration. The first electrode assembly includes a first spline assembly in a first planar loop in the first expanded configuration. A second electrode assembly is transitionable between a second expanded configuration and a second collapsed configuration when the first electrode assembly is in the first expanded configuration. The second electrode assembly having a second spline assembly in a second loop out of plane from the first planar loop in the second expanded configuration and in plane with the first planar loop in the second collapsed configuration.

### CROSS REFERENCE TO RELATED APPLICATIONS

This application claims priority to U.S. Provisional Application No. 63/686,013 entitled “CONFIGURABLE 2D TO 3D MAP AND ABLATE CATHETER,” filed Aug. 22, 2024, which is herein incorporated by reference in its entirety.

### TECHNICAL FIELD

The present disclosure relates to medical systems and methods for mapping an anatomical space of the body and ablating tissue in a patient. More specifically, the present disclosure relates to high density mapping and ablation catheters and systems and methods for cardiac, electroanatomical mapping and for ablation of tissue by electroporation such as in minimally invasive electrophysiological procedures.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

Electrophysiological procedures, which include catheter ablation to treat a variety of heart conditions such as supraventricular and ventricular arrhythmia, involve a visualization of the heart and heart activity. Electroanatomical mapping is a visualization technique that allows a clinician to accurately determine the location of an arrhythmia, define cardiac geometry in three dimensions, delineate areas of anatomic interest, and permits imaging of the catheter assembly for positioning and manipulation. For instance, electroanatomical mapping involves the mapping of electrical activity in the heart based on cardiac signals, such as at various locations on the endocardium surface, to identify the site of origin of the arrhythmia followed by a targeted ablation of the site.

To perform such cardiac mapping, a catheter with an electrode assembly at a distal tip of the catheter can be inserted into the patient's heart chamber. In some examples of mapping, physiological signals from electrical activity of the heart are acquired with electrodes after the tip is in stable and steady contact with the endocardium surface of a particular heart chamber. Alternatively, or additionally, physiological signals can be detected by non-contact electrodes along with information on chamber anatomy and relative electrode location to provide physiological information regarding the endocardium of the heart chamber. The locations of the physiological signals are determined, such as via location sensors on or near the electrode assembly. Location and electrical activity are measured, such as sequentially on a point-by-point basis in some examples, at about fifty to several hundred points on the internal surface of the heart to construct an electroanatomical map of the heart. The generated electroanatomical map can serve several purposes, such as the basis to decide on a therapeutic course of action like tissue ablation, which can be applied to alter the propagation of electrical activity in the heart and to restore normal heart rhythm.

### SUMMARY

In Example 1, a catheter to detect a plurality of physiological signals from within a patient's heart, the catheter comprising: an elongated shaft having a proximal region and a distal region, the elongated shaft defining a longitudinal axis; and a plurality of electrode assemblies extending distally from the distal region of the shaft and coupled together at a distal hub, the plurality of electrode assemblies including a first electrode assembly and a second electrode assembly; the first electrode assembly transitionable between a first electrode assembly expanded configuration and a first electrode assembly collapsed configuration, the first electrode assembly comprising: a first spline assembly configured in a first planar loop in the first electrode assembly expanded configuration, and a first plurality of electrodes disposed on the first spline assembly; the second electrode assembly transitionable between a second electrode assembly expanded configuration and a second electrode assembly collapsed configuration when the first electrode assembly is in the first electrode assembly expanded configuration, the second electrode assembly comprising: a second spline assembly coupled to the first spline assembly at the distal hub, the second spline assembly configured in a second loop out of plane from the first planar loop in the second electrode assembly expanded configuration and in plane with the first planar loop in the second electrode assembly collapsed configuration, and a second plurality of electrodes disposed on the second spline assembly.

In Example, 2, the catheter of Example 1, wherein the first electrode assembly in the first electrode assembly expanded configuration and the second electrode assembly in the second electrode assembly expanded configuration are transitionable to the first electrode assembly collapsed configuration and the second electrode assembly collapsed configuration.

In Example 3, the catheter of any of Examples 1 and 2, wherein the first electrode assembly in the first electrode assembly collapsed configuration and the second electrode assembly in the second electrode assembly collapsed configuration are transitionable to the first electrode assembly expanded configuration and the second electrode assembly expanded configuration.

In Example, 4, the catheter of any of Examples 1-3, wherein the plurality of electrodes includes a set of sensing electrodes and a set of ablation electrodes.

In Example 5, the catheter of any of Examples 1-4, wherein the first spline assembly in the first electrode assembly expanded configuration includes a first curved spline having a first distal end and an opposite, second curved spline having a second distal end, the first and second distal ends coupled to the distal hub.

In Example 6, the catheter of any of Examples 1-5, wherein the first spline assembly is configured from a nickel-titanium alloy.

In Example 7, the catheter of any of Examples 1-6, wherein the elongate shaft includes a transition member coupled to the second electrode assembly to transition the second electrode assembly between the second electrode assembly expanded configuration and the second electrode assembly collapsed configuration.

In Example 8, the catheter of Example 7, and further comprising a handle having a knob, wherein the knob is coupled to the transition member.

In Example 9, the catheter of Example 8, wherein the knob is configured to rotate about the longitudinal axis, and rotation of the knob transitions the second electrode assembly between the second electrode assembly expanded configuration and the second electrode assembly collapsed configuration.

In Example 10, the catheter of Example 8, wherein the knob is configured to translate along the longitudinal axis, and translation of the knob transitions the second electrode assembly between the second electrode assembly expanded configuration and the second electrode assembly collapsed configuration.

In Example 11, the catheter of any of Examples 1-10, wherein the second spline assembly includes a proximal end, and the proximal end is configured to be disposed within the elongated shaft in the second electrode assembly collapsed configuration and to slide out of the elongated shaft as the second electrode assembly transitions from the second electrode assembly collapsed configuration to the second electrode assembly expanded configuration.

In Example 12, the catheter of any of Examples 1-11, wherein the plurality of electrode assemblies includes one first electrode assembly and one second electrode assembly.

In Example 13, the catheter of any of Examples 1-11, wherein the plurality of electrode assemblies include a first electrode assembly and plurality of second electrode assemblies.

In Example 14, the catheter of Example 13, wherein the second electrode assemblies are each transitionable between a second electrode assembly expanded configuration and a second electrode assembly collapsed configuration when the first electrode assembly is in the first electrode assembly expanded configuration.

In Example 15, the catheter of any of Examples 13 and 14, wherein the plurality of electrode assemblies include one first electrode assembly and two second electrode assemblies.

In Example 16, a catheter to detect a plurality of physiological signals from within a patient's heart, the catheter comprising: an elongated shaft having a proximal region and a distal region, the elongated shaft defining a longitudinal axis; and a plurality of electrode assemblies extending distally from the distal region of the shaft and coupled together at a distal hub, the plurality of electrode assemblies including a first electrode assembly and a second electrode assembly; the first electrode assembly transitionable between a first electrode assembly expanded configuration and a first electrode assembly collapsed configuration, the first electrode assembly comprising: a first spline assembly configured in a first planar loop in the first electrode assembly expanded configuration, and a first plurality of electrodes disposed on the first spline assembly; the second electrode assembly transitionable between a second electrode assembly expanded configuration and a second electrode assembly collapsed configuration when the first electrode assembly is in the first electrode assembly expanded configuration, the second electrode assembly comprising: a second spline assembly coupled to the first spline assembly at the distal hub, the second spline assembly configured in a second loop out of plane from the first planar loop in the second electrode assembly expanded configuration and in plane with the first planar loop in the second electrode assembly collapsed configuration, and a second plurality of electrodes disposed on the second spline assembly.

In Example 17, the catheter of Example 16, wherein the first electrode assembly in the first electrode assembly expanded configuration and the second electrode assembly in the second electrode assembly expanded configuration are transitionable to the first electrode assembly collapsed configuration and the second electrode assembly collapsed configuration.

In Example 18, the catheter of Example 16, wherein the first electrode assembly in the first electrode assembly collapsed configuration and the second electrode assembly in the second electrode assembly collapsed configuration are transitionable to the first electrode assembly expanded configuration and the second electrode assembly expanded configuration.

In Example 19, the catheter of Example 16, wherein the plurality of electrodes includes a set of sensing electrodes and a set of ablation electrodes.

In Example 20, the catheter of Example 16, wherein the first spline assembly in the first electrode assembly expanded configuration includes a first curved spline having a first distal end and an opposite, second curved spline having a second distal end, the first and second distal ends coupled to the distal hub.

In Example 21, the catheter of Example 16, wherein the first spline assembly is configured from a nickel-titanium alloy.

In Example 22, the catheter of Example 16, wherein the elongate shaft includes a transition member coupled to the second electrode assembly to transition the second electrode assembly between the second electrode assembly expanded configuration and the second electrode assembly collapsed configuration.

In Example 23, the catheter of Example 22, and further comprising a handle having a knob, wherein the knob is coupled to the transition member.

In Example 24, the catheter of Example 23, wherein the knob is configured to rotate about the longitudinal axis, and rotation of the knob transitions the second electrode assembly between the second electrode assembly expanded configuration and the second electrode assembly collapsed configuration.

In Example 25, the catheter of Example 23, wherein the knob is configured to translate along the longitudinal axis, and translation of the knob transitions the second electrode assembly between the second electrode assembly expanded configuration and the second electrode assembly collapsed configuration.

In Example 26, the catheter of Example 16, wherein the second spline assembly includes a proximal end, and the proximal end is configured to be disposed within the elongated shaft in the second electrode assembly collapsed configuration and to slide out of the elongated shaft as the second electrode assembly transitions from the second electrode assembly collapsed configuration to the second electrode assembly expanded configuration.

In Example 27, the catheter of Example 16, wherein the plurality of electrode assemblies includes one first electrode assembly and one second electrode assembly.

In Example 28, the catheter of Example 16, wherein the plurality of electrode assemblies include a first electrode assembly and plurality of second electrode assemblies.

In Example 29, the catheter of Example 28, wherein the second electrode assemblies are each transitionable between a second electrode assembly expanded configuration and a second electrode assembly collapsed configuration when the first electrode assembly is in the first electrode assembly expanded configuration.

In Example 30, the catheter of Example 28, wherein the plurality of electrode assemblies include one first electrode assembly and two second electrode assemblies.

In Example 31, a catheter to detect a plurality of physiological signals from within a patient's heart, the catheter comprising: an elongated shaft having a proximal region and a distal region, the elongated shaft defining a longitudinal axis; and a plurality of electrode assemblies extending distally from the distal region of the shaft and coupled together at a distal hub, the plurality of electrode assemblies including a first electrode assembly and a second electrode assembly, wherein the plurality of electrodes includes a set of sensing electrodes and a set of ablation electrodes; the first electrode assembly transitionable between a first electrode assembly expanded configuration and a first electrode assembly collapsed configuration, the first electrode assembly comprising: a first spline assembly configured in a first planar loop in the first electrode assembly expanded configuration, and a first plurality of electrodes disposed on the first spline assembly; the second electrode assembly transitionable between a second electrode assembly expanded configuration and a second electrode assembly collapsed configuration when the first electrode assembly is in the first electrode assembly expanded configuration, the second electrode assembly comprising: a second spline assembly coupled to the first spline assembly at the distal hub, the second spline assembly configured in a second loop out of plane from the first planar loop in the second electrode assembly expanded configuration and in plane with the first planar loop in the second electrode assembly collapsed configuration, and a second plurality of electrodes disposed on the second spline assembly.

In Example 32, the catheter of Example 31, wherein the first electrode assembly in the first electrode assembly expanded configuration and the second electrode assembly in the second electrode assembly expanded configuration are transitionable to the first electrode assembly collapsed configuration and the second electrode assembly collapsed configuration.

In Example 33, the catheter of Example 32, wherein the first electrode assembly in the first electrode assembly collapsed configuration and the second electrode assembly in the second electrode assembly collapsed configuration are transitionable to the first electrode assembly expanded configuration and the second electrode assembly expanded configuration.

In Example 34, a system to detect a plurality of physiological signals from within a patient's heart, the system comprising: an electroporation console configured to deliver electric field energy to targeted tissue in the patient's heart, the electroporation console coupled to a catheter, the catheter comprising: an elongated shaft having a proximal region and a distal region, the elongated shaft defining a longitudinal axis; and a plurality of electrode assemblies extending distally from the distal region of the shaft and coupled together at a distal hub, the plurality of electrode assemblies including a first electrode assembly and a second electrode assembly, wherein the plurality of electrodes includes a set of sensing electrodes and a set of ablation electrodes, the electric field energy delivered to the set of ablation electrodes; the first electrode assembly transitionable between a first electrode assembly expanded configuration and a first electrode assembly collapsed configuration, the first electrode assembly comprising: a first spline assembly configured in a first planar loop in the first electrode assembly expanded configuration, and a first plurality of electrodes disposed on the first spline assembly; the second electrode assembly transitionable between a second electrode assembly expanded configuration and a second electrode assembly collapsed configuration when the first electrode assembly is in the first electrode assembly expanded configuration, the second electrode assembly comprising: a second spline assembly coupled to the first spline assembly at the distal hub, the second spline assembly configured in a second loop out of plane from the first planar loop in the second electrode assembly expanded configuration and in plane with the first planar loop in the second electrode assembly collapsed configuration, and a second plurality of electrodes disposed on the second spline assembly.

In Example 35, the system of Example 34, and further comprising an electroanatomical mapping system coupled to the set of sensing electrodes.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2 is perspective view of an embodiment of a catheter for use with the electrophysiology system of FIG. 1.

FIG. 3A is a perspective view of a distal end of the catheter of FIG. 2 in a fully collapsed configuration.

FIG. 3B is a perspective view of the distal end of the catheter in FIG. 2 in a partially collapsed (partially expanded) configuration.

FIG. 3C is a perspective view of the distal end of the catheter of FIG. 2 in a fully expanded configuration.

FIG. 4A is a perspective view of a distal end of another embodiment of a catheter for use with the electrophysiology system of FIG. 1 in a fully expanded configuration.

FIG. 4B is a perspective view of the distal end of the catheter in FIG. 4A in a partially collapsed (partially expanded) configuration.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

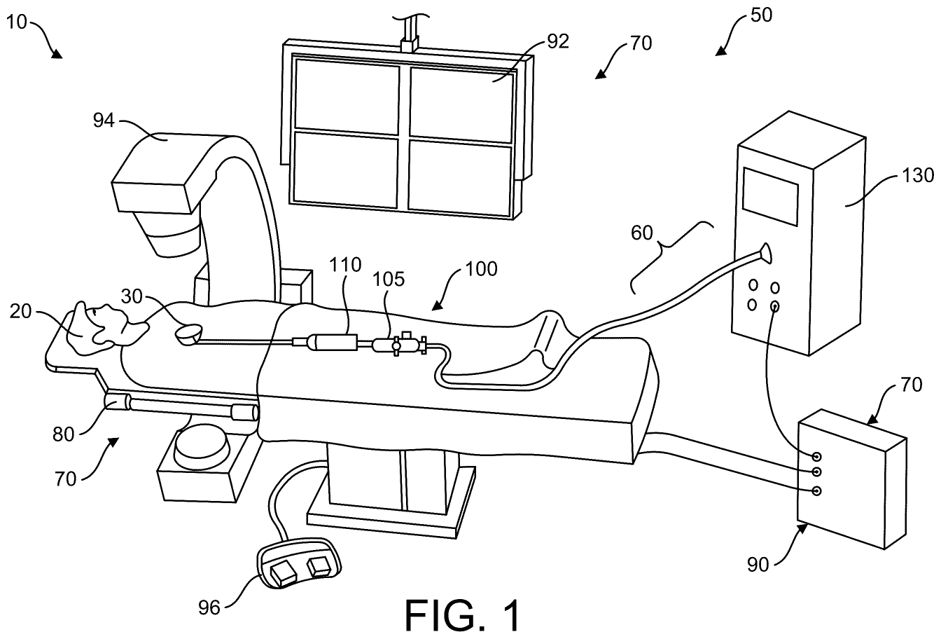

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1. Other arrangements of connecting elements, including wireless connecting elements, are contemplated.

The electroporation catheter system 60 is configured to deliver electric field energy to targeted tissue in the patient's heart 30 to create cell death in tissue, for example, rendering the tissue incapable of conducting electrical signals. Also, the electroporation catheter system 60 is configured to generate, based on models of electric fields, graphical representations of the electric fields that can be produced using the electroporation catheter 105 and to overlay, on the display 92, the graphical representations of the electric fields or expected or predicted lesions on an anatomical map of the patient's heart to aid a user in planning ablation by irreversible electroporation using the electroporation catheter 105 prior to delivering energy. In embodiments, the electroporation catheter system 60 is configured to generate the graphical representations of the electric fields based on characteristics of the electroporation catheter 105 and the position of the electroporation catheter 105 in the patient 20, such as in the heart 30 of the patient 20. The electroporation catheter system 60 is configured to generate the graphical representations of the electric fields based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

The example electroporation catheter 105 includes an elongated catheter shaft and distal end configured to be deployed proximate target tissue, such as within a chamber of the patient's heart. The distal end includes an electrode assembly to effect treatment. The catheter 105 is capable of being formed into a plurality of configurations. For example, if the distal end region of the catheter is within the patient's vasculature or is within a sheath as a catheter assembly, such as to travel to the patient to the chamber of the heart, the electrode assembly is in a collapsed configuration to fit within the sheath. Once the catheter has reached the destination in the chamber of the heart, for example, or the sheath is retracted from the distal region of the catheter 105 (or the shaft catheter is extended past the sheath), and the electrode assembly is arranged in an expanded configuration for use. In one embodiment, the electrode assembly can assume other configurations, such as an intermediate configuration between the collapsed and expanded configurations, such as an additional use configuration.

The electrode assembly includes an electrode assembly comprising a plurality of electrodes. For example, the electrode assembly includes a plurality of spaced-apart electrodes or multiple spaced-apart sets or groups of spaced-apart electrodes. In some examples, an electrode, such as a plurality of spaced-apart electrodes, can be deployed on the catheter shaft in addition to electrodes on the electrode assembly. In one example, the plurality of electrodes can be formed of a conductive, solid-surface, biocompatible material and are spaced-apart across electrical insulators. Each of the plurality of electrodes is electrically coupled to an associated elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be electrically insulated from one another within an electrically insulating sheath along the catheter shaft, such as with an electrically insulating polymer sheath. The lead conductors can be electrically coupled to plug in the proximal region of the electroporation catheter 105, such as a plug configured to be mechanically and electrically coupled to the electroporation console 130 and the EAM system 70, for example, either directly or via intermediary electrical conductors such as cabling.

The electrode assemblies and associated electrodes are configured for, among other things, sensing cardiac electrical signals, ablation, localization of the electrode assembly within the patient anatomy such as via the EAM system 70, signal reference, and to determine proximity to target tissue within the anatomy. In some embodiments, the catheter 105 is configured for cardiac mapping, and the electrodes are sensing, or mapping, electrodes configured to be used to collect physiological (electrical) signals to be used to generate electroanatomical maps. An example of a physiological signal that the sensing electrode can acquire includes an intracardiac electrogram (ECG) signal. In some embodiments, the catheter 105 can be a mapping and ablation catheter, and the electrodes can include ablation electrodes, or an ablation electrode assembly, that are configured to deliver ablation electric field energy and sensing electrodes, or a sensing electrode assembly, for mapping purposes. The ablation electrodes in embodiments of an electroporation catheter are configured to receive pulsed electrical signals or waveforms from the console 130 and create pulsed electric fields sufficient to ablate target tissue via irreversible electroporation. The sensing electrodes in the electrode assembly can be electrically coupled to a one or more lead conductors that extends the length of the shaft that are configured to carry an electrical signal received at the sensing electrode. In some examples, an electrode in the electrode assembly can be configured to only perform an ablation or the electrode in the electrode assembly can be configured to only perform mapping. In some examples, an electrode can operate as an ablation electrode in an ablation mode of the electrophysiology system 50 and as a sensing electrode in a sensing or mapping mode of the system 50. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, to effect electroporation.

A selected electrical field can be generated with the electrodes configured as ablation electrodes to effect electroporation. A first ablation electrode, or first group of ablation electrodes, can be selected to be an anode and a different, second ablation electrode, or second group of ablation electrodes, can be selected to be a cathode, such that electrical fields can be generated between the anode and cathode based on signals, such as pulses, provided to the ablation electrodes from the electroporation console 130. The console 130 provides electric pulses of different lengths and magnitudes to the ablation electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more ablation electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an ablation electrode, a group of ablation electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly. For example, the electrodes on the ablation electrode assembly are configured as the one of the anode or cathode and electrodes on the shaft proximate the distal end are configured as the other of the cathode or anode.

The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the OPAL™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the OPAL HDx™ mapping system.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

Each cardiac physiological (electrical) signal can include several intracardiac electrograms (EGMs) sensed within a patient's heart and may include any number of features that may be ascertained by aspects of the system 50. Examples of cardiac physiological signal features include activation times, activations, activation waveforms, filtered activation waveforms, minimum voltage values, maximum voltages values, maximum negative time-derivatives of voltages, instantaneous potentials, voltage amplitudes, dominant frequencies, and peak-to-peak voltages. A cardiac physiological signal feature can refer to one or more features extracted from one or more cardiac physiological signals, derived from one or more features that are extracted from one or more cardiac physiological signals. Additionally, a representation, on a cardiac or a surface map, of a cardiac physiological signal feature may represent one or more cardiac physiological signal features, an interpolation of several cardiac physiological signal features. Each cardiac physiological signal also can be associated with a set of respective position coordinates that corresponds to the location at which the cardiac physiological signal was sensed. Each of the respective position coordinates for the sensed cardiac physiological signals can include three-dimensional Cartesian coordinates, polar coordinates, or another coordinate system. The cardiac physiological signals may be sensed on the cardiac surfaces, and the respective position coordinates can be on the endocardial surface, epicardial surface, in the mid-myocardium of the patient's heart, or in a vicinity.

During a signal-acquisition stage of a cardiac mapping procedure, the catheter 105 is displaced to multiple locations within the heart chamber into which the catheter 105 is inserted. At each location to which the catheter 105 is moved, the electrodes and sensors acquire physiological signals resulting from the electrical activity in the heart along with positional, or spatial, information of the catheter 105. The spatial information is used in building a three-dimensional grid of the anatomy during mapping. To perform a mapping procedure and reconstruct physiological information on the endocardium surface, the EAM system 70 may align a coordinate system of the catheter 105 with the endocardium surface's coordinate system, or vice versa. Alternatively, or additionally, the grid may be used to capture EGMs, and select mapping values based on statistical distributions associated with nodes of the grid. The EAM system 70 also can perform post-processing operations on the physiological information to extract and display useful features of the information to the operator of the system 50.

In generating an example electroanatomical map, a data stream including multiple signals, such as signals received from the mapping electrodes of the catheter 105, is input into the EAM system 70. During the automated electroanatomical mapping process, the data stream provides a collection of physiological and location signals that serve as an input to the mapping process. The signals may be collected directly by the mapping system, obtained from another system using an analog or digital interface, or both. The data stream can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data of the cardiac surface is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry of the cardiac surface can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

Many tools available for electrophysiology procedures, such as mapping and ablation catheters, include a single distal configuration or form factor, which is used to address multiple cardiac chambers or cardiac anatomy. Tools that work well in atria, however, may not be suitable for ventricles, and vice versa. In one instance, a catheter that is of a configuration suited for a particular mapping of cardiac anatomy may not be suited for ablation of a selected region within the anatomy. Additionally, tools that lack versatility also present issues for mapping and ablation procedures in that clinicians often employ mapping catheters of a particular or applicable configuration and then switch to a separate ablation catheter of a suitable configuration. The use of separate catheters for mapping and ablation often includes multiple product exchanges through a delivery sheath to the heart, which can result in a higher risk of air ingress, procedure times, and cost of the procedure. Some combination mapping and ablation catheters also present issues in catheter handling, stiffness influence on mapping, navigation in smaller cardiac regions, and cost of the catheter.

The embodiments of the catheter set out a distal region convertible into multiple shapes, sizes, and form factors. In embodiments, the electrode assemblies of the catheter can convert from a two-dimensional shape, such as a paddle type mapping catheter to a three-dimensional shape, such as in a basket type mapping and ablation catheter. A clinician can readily convert the configurations via controls on the handle, such as via tension and compression actuation or via torque actuation, and without product exchanges. In one embodiment, each of the multiple configurations are suitable for use in mapping and ablation.

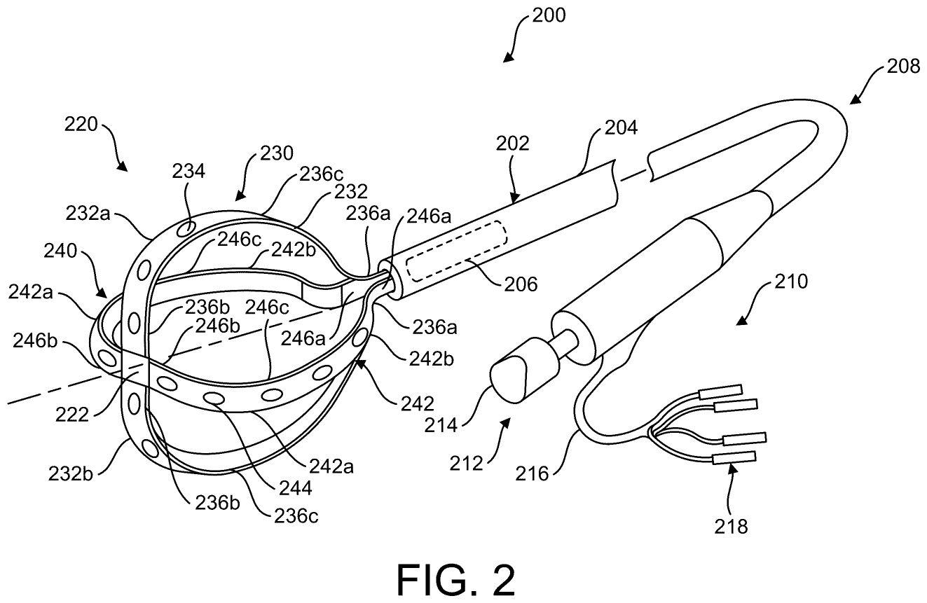

FIG. 2 is a partial perspective illustration of an embodiment of a catheter 200 configured to detect a plurality of physiological signals from within a patient's heart. The catheter 200 corresponds with the catheter 105 described with respect to FIG. 1 and can be used in the electrophysiology system 50. For example, catheter 200 can be configured as a mapping catheter in some embodiments and as a mapping and ablation catheter via irreversible electroporation in some embodiments. The catheter 200 has an elongated shaft 202 having a proximal region 208 and a shaft distal region 204 configured to be deployed proximate target tissue, such as within a chamber of the patient's heart. The elongated shaft 202 defines a longitudinal axis A, which is presented as a line passing through a centroid of a cross section of the shaft 202. In the illustrated embodiment, the catheter 202 includes a location sensor 206 (shown in phantom) coupled to the distal region 204, such as within the shaft 202, in which the location sensor 206 can be configured to generate a plurality of location signals representative of a location of the catheter 200 within the patient's heart. A plurality of electrode assemblies 220 extend distally from the distal region 204 of the shaft 204. The plurality of electrode assemblies 220 are coupled together at a distal hub 222.

In embodiments, the elongate shaft 202 is formed of a biocompatible material that provide sufficient sturdiness and flexibility to allow the shaft 202 to be navigated through the vasculature of a patient and reach the treatment site, such as a chamber of the heart. In some embodiments, the shaft 202 is formed of multiple different materials to provide the catheter 200 with more flexibility at the distal region 204 than the proximal region 208. Further, the shaft 202 can included a tubular woven member to provide torsional stiffness and bending flexibility. The shaft 202 can include various markers for use with a visualization system, such as radiopaque or echogenic markers, or EAM electrodes to facilitate visualization and location. The catheter shaft 202 can also accommodate a transition device such as pull wires or other mechanisms to direct, bend, twist, or tilt the electrode assemblies 220. The pull wires or other mechanisms can also serve to select or adjust the angle of the electrode assemblies 220 with respect to the shaft 202, and the relationship of the electrode assemblies 220 to the shaft 202 can be adjusted. The distal region 204 can include sensors such as one or more tracking, or location sensors 206 (shown in phantom). The location sensor 206 can include a magnetic sensor for tracking the location of the catheter 200 within a magnetic location field or an EAM electrode for tracking the location of the catheter 200 within an electric location field. In the illustrated embodiment, the location sensor 206 is a magnet location sensor disposed within the shaft 202. In other embodiments, the location sensor 206 can be disposed on the electrode assemblies 220. The distal region 204 can also include force sensors and additional elements such as an irrigation element. In some embodiments, a stem is included extending from the elongate shaft 202, and the stem is configured to include irrigation elements and sensors and other components.

The proximal region 208 of the catheter shaft 202 in some embodiments is coupled to a handle 210. The handle 210 is operably coupled to the electrode assemblies 220. For instance, the handle 210 includes a translation member 212 coupled to the transition device such as the pull wires or other mechanisms. The translation member 212, in some embodiments, includes a knob 214 configured to be manipulated translationally or rotationally by a clinician. For example, the translation member 212 is configured for translation along or rotation about the longitudinal axis to transition one or more electrode assemblies of the plurality of electrode assemblies 220 between a set of configurations including a fully collapsed configuration, a partially collapsed configuration (or partially extended configuration), and a fully expanded configuration. A clinician can push or pull on the knob 214 to actuate the translation member 212 in some embodiments to transition the electrode assemblies, or the clinician can apply a torque to the knob 214 to actuate the translation member 212 in some embodiments. As an additional example, the translation member 212 can be configured to be manipulated to transition the catheter 200 between a lock state and an unlock state. The lock state can fix a configuration of one or more electrode assemblies of the plurality of the electrode assemblies 220 relative to the catheter shaft 202 and the unlock state can permit translation or rotation of one or more of the electrode assemblies of the plurality of electrode assemblies 220 relative to the catheter shaft 202. In some embodiments, the handle 210 includes a flush port in communication with the distal region 204 to receive an irrigation fluid such as saline. The handle 210 also includes an electrical cable 216 having one or more electrical connectors 218. The electrical connectors 218 are configured to couple to an extension cable to connect the catheter 200 to an electrophysiological component such as the console 130 or the mapping and navigation controller 90, for example The connectors 218 are electrically connected to electrical components such as the electrodes, impedance tracking sensors, a magnetic sensor, temperature sensor, a gyroscopic sensor, an accelerometer via leads disposed in the handle 210, shaft 202, or on the plurality of electrode assemblies 220.

In the illustrated embodiment, the plurality of electrode assemblies 220 include a first electrode assembly 230 and a second electrode assembly 240. The first electrode assembly 230 includes a first spline assembly 232. A first plurality of electrodes 234 are disposed on the first spline assembly 232. In one embodiment, the first spline assembly 232 includes a pair of first splines 232a, 232b, such as a pair of opposing first splines. The opposing first splines 232a, 232b are configured such that the first spline assembly 232 forms a planar loop in the expanded configuration. Each of the first splines 232a, 232b includes a first spline proximal region 236a, a first spline distal region 236b, and a first spline intermediate portion 236c. The first spline proximal regions 236a of the pair of first splines 232a, 232b are coupled to the shaft distal region 204. In one embodiment, the first spline proximal regions 236a are coupled to the shaft distal region 204 in such as manner so as not to longitudinally translate with respect to the shaft 202 as the catheter 202 transitions between the fully collapsed configuration and the fully expanded configuration. The first spline distal regions 236b are coupled together at the distal hub 222, and the first spline intermediate portions 236c extend longitudinally in the fully collapsed configuration and are laterally spaced apart from each other in the fully expanded configuration. In one embodiment, the first spline distal regions 236b are integrated together. In the illustrated embodiment, some or all electrodes of the first plurality of electrodes 234 are disposed on the first spline intermediate portion 236c.

The second electrode assembly 240 includes a second spline assembly 242 coupled to the first spline assembly 232 at the distal hub 222. A second plurality of electrodes 244 are disposed on the second spline assembly 242. In one embodiment the second spline assembly 242 includes at least a pair of second splines 242a, 242b, such as pair of opposing second splines. Two second splines 242a, 242b formed as a second planar loop are shown in the illustrated embodiment, although the second electrode assembly 240 can be configured to include any number of one or more second splines, such as four second splines. Each of the second splines 242a, 242b includes a second spline proximal region 246a, a second spline distal region 246b, and a second spline intermediate portion 246c. The second spline proximal regions 246a of the pair of second splines 242a, 242b are proximate to the shaft distal region 204. In one embodiment, the second spline proximal regions 246a can translate along the longitudinal axis, such as within a tubular portion of the shaft distal region 204 as the catheter 202 transitions between the fully collapsed configuration and a partially collapsed or partially expanded configuration. For instance, the second spline proximal region 246a can slide into the shaft distal region 204 as the catheter 202 transitions from a fully expanded configuration to a partially collapsed configuration, and the second spline proximal region 246a can slide out of the shaft distal region 204 as the catheter 202 transitions from a the partially collapsed configuration to either the fully expanded configuration or the fully collapsed configuration. In one embodiment, the translation member 212 is actuated to transition the second electrode assembly between the fully expanded configuration and the partially collapsed configuration. The second spline distal regions 246b are coupled to the distal hub 222, and the second spline intermediate portions 246c extend longitudinally in the fully collapsed configuration and are laterally spaced apart from each other in the fully expanded configuration. In one embodiment, the second spline distal regions 246b are integrally formed together. In the illustrated embodiment, some or all electrodes of the second plurality of electrodes 244 are disposed on the first spline intermediate portion 246c.

The first and second spline assemblies 232, 234 are configured to transition between configurations. In one embodiment, the splines are constructed to include a polyether block amide and, in some examples, is available under the trade designations PEBAX from Arkema, S.A., and VESTAMID E from Evonik Industries, AG. In some embodiments, each spline defines a spline lumen extending along a length of spline and closed at the distal hub 222 or by the distal cap. Electrical leads or electrical components can be disposed within the spline lumen such as in a manner that permits bending, extending, or translating the splines in the set of splines. In some embodiments, the splines are constructed to include a superelastic material (metal or polymer) to provide desired mechanical/structural properties to the one or both of the electrode assemblies 220. In embodiments, one or both of the first and second spline assemblies 232, 242 are formed from a superelastic metal alloy, e.g., a nickel-titanium alloy or nitinol. In one embodiment, the first spline assembly 232 is formed of a nickel-titanium alloy and the second spline assembly 234 is formed of a polymer. Forming the splines from a superelastic material such as a nickel-titanium alloy facilitates configuring the splines to assume its desired unconstrained shape due to the shape memory properties of the material, while providing sufficient flexibility necessary to collapse or expand the electrode assemblies 220. The shape memory splines predictably governs the shape of the electrode array 202 through transition between the fully collapsed configuration, partially collapsed configuration, and fully expanded configuration. Further, in some examples, the shape memory spline can serve to reduce deployment force during transitions. In one embodiment, the shape memory first and second spline assemblies 232, 242 are shape set in the expanded configuration such as to form a basket. In another embodiment, the shape memory first spline assembly 232 is shape set in the expanded configuration such as to form a loop and the second spline assembly 242 is shape memory spline assembly is shape set in the collapsed configuration. In still another embodiment, the shape memory first spline assembly is shape set in the expanded configuration such as to form a loop and the second spline assembly does not include a superelastic material. Other combinations are contemplated.

Electrodes 234, 244 are disposed on the splines 232, 242, respectively, such that each spline in the first and second electrode assemblies 230, 240 includes a set of outwardly-facing electrodes. Electrodes 234, 244 can be configured as dedicated ablation electrodes, dedicated sensing electrodes, or electrodes configured for ablation and sensing. In one embodiment, each spline of the first and second electrode assemblies 230, 240 includes ablation electrodes and sensing electrodes, or electrodes configured for ablation and sensing. In some embodiments, each spline includes a set of electrodes having from two electrodes to eight electrodes, or more. In embodiments, each electrode is spaced-apart from other electrodes on that spline and on other splines in the fully expanded configuration. The electrodes include an atraumatic shape to reduce trauma to tissue. For example, the electrodes have an atraumatic shape including a rounded, flat, curved, or blunted portion configured to contact endocardial tissue. In some embodiments, the electrodes may be located along any portion of the spline distal to the catheter shaft 202. In some embodiments, an additional electrode, such as an additional electrode used for ablation, can be located on the shaft distal region 204, such as to operate the catheter in a bipolar mode. For instance, the addition electrode, which can include a plurality of additional electrodes on the shaft 202, can be operated as one of the cathode or anode when electrodes on the plurality of electrode assemblies 220 are operated as the other of the anode or cathode such as for ablation. The electrodes can have the same or different sizes, shapes, and/or location along respective splines.

In some embodiments, ablation electrodes are electrically coupled to insulated electrical leads (not sown), such as each spline in the set of splines is associated with an electrical lead, such as an electrically insulated electrical lead, electrically coupled to the ablation electrode on that spline. For example, each ablation electrode on that spline can be associated with one electrical lead, which is electrically coupled to each ablation electrode. In some embodiments, one electrical lead is coupled to one or more ablation electrodes, and each ablation electrode can be associated with one connected lead in some embodiments. In some embodiments, the electrical lead is disposed within the spline lumen. In some embodiments, the electrical lead is disposed on the surface of the spline. In one instance, the electrical leads are included as lead traces on a flexible circuit. In some embodiments, the set of ablation electrodes for each spline are jointly wired (whether connected by wires or conductive lead traces). In some embodiments, the set of ablation electrodes for each spline are wired in series. For example, a set of four electrodes on a spline are electrically coupled together using a single lead. The electrical lead may be disposed within a spline lumen to electrically couple to an associated ablation electrode through a corresponding aperture in the spline. In one embodiment, each electrical lead coupled to an electrode configured for ablation is capable of sustaining a voltage potential of at least about 700 Volts without dielectric breakdown of the corresponding insulation.

In some embodiments, the electrodes are independently addressable, and the electrodes configured for ablation can be energized in any sequence using any pulse waveform sufficient to ablate tissue by irreversible electroporation. For example, different sets of electrodes can deliver different sets of pulses (e.g., hierarchical pulse waveforms). The size, shape, and spacing of the ablation electrodes on each spline and between the splines are configured to deliver contiguous/transmural energy to electrically isolate one or more pulmonary veins in some embodiments. In some embodiments, a first set of electrodes of a spline or splines is configured as an anode and a second set of electrodes of another spline or splines is configured as a cathode. The spline can be non-adjacent to the another spline. This can increase the spacing between the splines and help prevent a short-circuit. In some of these embodiments, the first set of electrodes includes one electrode configured for ablation and the second set of ablation electrodes includes at least two electrodes configured for ablation. In some embodiments, alternate electrodes are at the same electric potential, and likewise for all the other alternating ablation electrodes. Thus, ablation may be delivered rapidly with all ablation electrodes activated at the same time. For example, a spline having a set of anode electrodes can be activated together to deliver pulse waveforms for irreversible electroporation. Electrodes on other splines can be activated together as cathode ablation electrodes on their respective splines so as to form an anode-cathode pairing for delivery of pulse waveforms for irreversible electroporation. The anode-cathode pairing and pulse waveform delivery can be repeated sequentially over a set of such pairings. For example, the splines can be activated sequentially in a clockwise or counter-clockwise manner. As another example, the cathode splines can be activated sequentially along with respective sequential anode spline activation until ablation is completed. In embodiments where electrodes on a given spline are wired separately, the order of activation within the electrode of each spline can be varied as well. For example, the electrodes in a spline can be activated all at once or in a predetermined sequence.

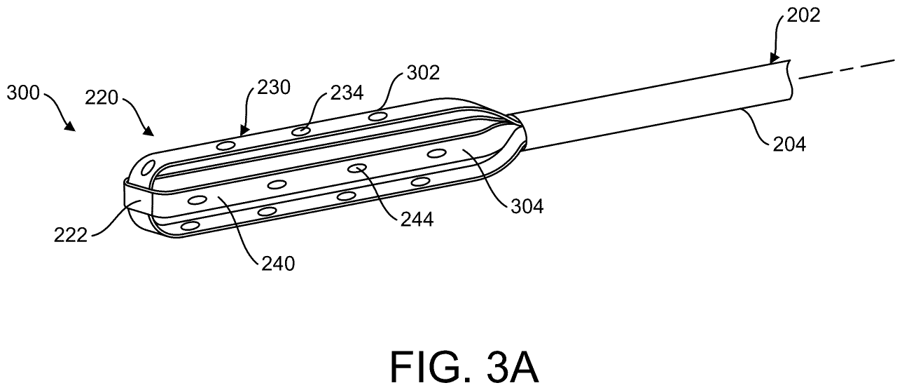

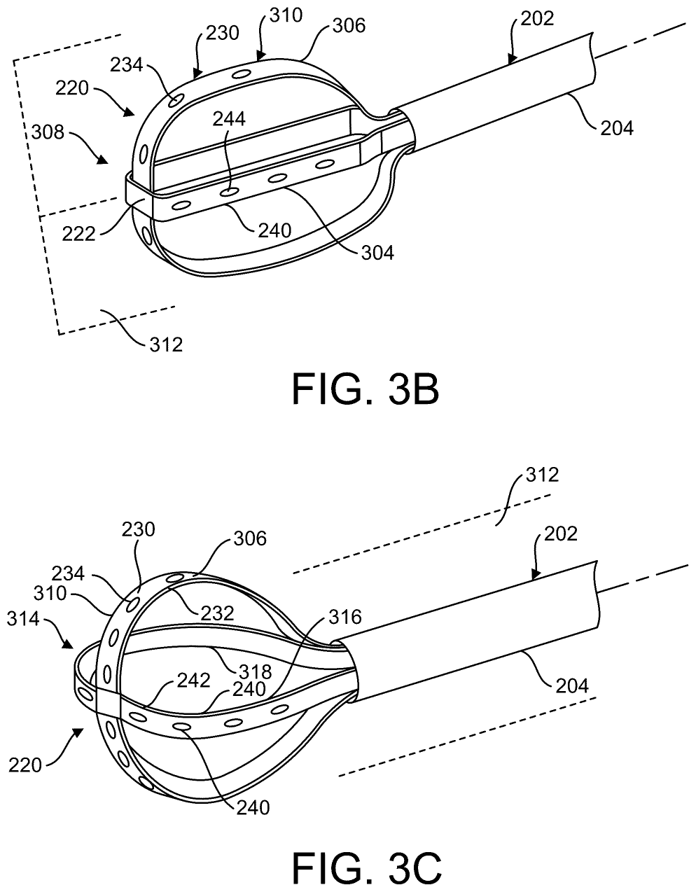

FIGS. 3A-3C illustrate the plurality of electrode assemblies 220 of catheter 200 in which the catheter is in a plurality of configurations. FIG. 3A illustrates the plurality of electrode assemblies 220 for the catheter 200 in the collapsed configuration 300. In particular, the first electrode assembly 230 is in a first electrode assembly collapsed configuration 302 and the second electrode assembly 240 is in a second electrode assembly collapsed configuration 304. The plurality of electrode assemblies 220 having first electrode assembly 230 in the first electrode assembly collapsed configuration 302 and the second electrode assembly 240 in the second electrode assembly collapsed configuration 304 are defined in a longitudinally extending cylinder. The profile of the plurality of electrode assemblies 220 are such that the catheter 200 can be advanced through a patient's vasculature. For instance, the plurality of electrode assemblies can be disposed within a delivery component such as an introducer sheath. In one embodiment, the first electrode assembly 230 is shape set into a first electrode assembly expanded configuration and collapsed by the sheath. And the second electrode assembly 240 can be shape set into a second electrode assembly expanded configuration and partially translated into a lumen in the shaft 202 via the translation member 212 to the second electrode assembly collapsed configuration or partially translated into a lumen in the shaft 202 via urging against the sheath.

FIG. 3B illustrates the plurality of electrode assemblies 220 of the catheter 200 in a partially expanded configuration or a partially collapsed configuration 308. In particular, the first electrode assembly 230 is in a first electrode assembly expanded configuration 306 and the second electrode assembly 240 is in a second electrode assembly collapsed configuration 304. The first electrode assembly 230 in the first electrode assembly expanded configuration 306 is defined as a first planar loop 310, and the second electrode assembly 240 in the second electrode assembly collapsed configuration 304 is defined as a longitudinally extending cylinder in the plane 312 of the first electrode assembly 230. For instance, the second spline assembly 242 is in plane 312 with the first spline assembly 232. The proximal ends 246a of the second spline assembly 242 are disposed within the lumen of the shaft 202 such that the second spline assembly 242 is not fully extended from the shaft 202. Accordingly, the partially expanded or partially collapsed configuration 308 defines a two-dimensional paddle shape configured in the plane 312. Electrodes configured for sensing or the sensing electrodes disposed on the plurality of electrode assemblies are exposed and distal to the shaft 202. In some embodiments, some or all electrodes configured for ablation or ablation electrodes are disposed within the shaft lumen in the shaft distal region 204.

FIG. 3C illustrates the plurality of electrode assemblies 220 of the catheter 200 in a fully expanded configuration 314. In particular, the first electrode assembly 230 is in the first electrode assembly expanded configuration 306 and the second electrode assembly 240 is in a second electrode assembly expanded configuration 316. The first electrode assembly 230 in the first electrode assembly expanded configuration 306 is defined as the first planar loop 310, and the second electrode assembly 240 in the second electrode assembly expanded configuration 316 is defined as a second loop 318 out of the plane 312 of the first electrode assembly 230. For instance, the planar loop 318 of the second spline assembly 242 forms second planar loop 318 in the illustrated embodiment. In embodiments in which the second electrode assembly 242 includes multiple loops from four or more second splines, each second planar loop is out of plane of the first planar loop 310 of the first spline assembly 232. Accordingly, the fully expanded configuration 314 defines a three-dimensional basket shape. In the second electrode assembly expanded configuration 316, the second spline assembly 242 is extended from the shaft lumen in the shaft distal region 204. Electrodes configured for sensing or the sensing electrodes disposed on the plurality of electrode assemblies 220 are exposed and distal to the shaft 202. Also, electrodes configured for ablation or the ablation electrodes disposed on the plurality of electrode assemblies 220 are exposed and distal to the shaft 202.

In embodiments, the plurality of the electrode assemblies 220 in the fully collapsed configuration 300 of FIG. 3A can transition to a two-dimensional partially expanded configuration 308 of FIG. 3B or to the three-dimensional fully expanded configuration 314 of FIG. 3C. In particular, the first electrode assembly 230 in the first electrode assembly collapsed configuration 302 can transition to the first electrode assembly expanded configuration 302 and the second electrode assembly 240 can remain in the second electrode collapsed configuration 304 and not transition to obtain the two-dimensional partially expanded configuration 308 of FIG. 3B. Also, the first electrode assembly 230 in the first electrode assembly collapsed configuration 302 can transition to the first electrode assembly expanded configuration 302 and the second electrode assembly 340 in the second electrode collapsed configuration 304 can transition to the second electrode expanded configuration 316 to put the plurality of electrode assemblies 220 into the fully expanded configuration 314 of FIG. 3C.

The plurality of electrode assemblies 220 in the two-dimensional partially expanded or partially collapsed configuration 308 of FIG. 3B can transition into the fully collapsed configuration 300 of FIG. 3A or to the three-dimensional fully expanded configuration 314 of FIG. 3C. In the partially expanded configuration 308 of FIG. 3B, the first electrode assembly 230 in the first electrode assembly expanded configuration 306 can transition to the first electrode assembly collapsed configuration 302 and the second electrode assembly 240 can remain in the in the second electrode assembly collapsed configuration 304 and not transition to put the plurality of electrodes 220 in the fully collapsed configuration 300 of FIG. 3A. Also, in the partially expanded configuration 308 of FIG. 3B, the first electrode assembly 230 can remain in the first electrode expanded configuration 306 and not transition, and the second electrode assembly 240 in the second electrode assembly collapsed configuration 304 can transition to the second electrode assembly expanded configuration 316 to put the plurality of electrode assemblies 220 in a fully expanded configuration 314 of FIG. 3C.

The plurality of electrode assemblies 220 in the three-dimensional fully expanded configuration 314 of FIG. 3C can transition into the two-dimensional partially collapsed configuration 308 of FIG. 3B or to the fully collapsed configuration 300 of FIG. 3A. In the plurality of electrode assemblies 220 in the three-dimensional fully expanded configuration 314 of FIG. 3C, the first electrode assembly 230 can remain in the first electrode expanded configuration 306 and not transition, and the second electrode assembly 240 in the second electrode expanded configuration 316 can transition to the second electrode collapsed configuration 304 to put the plurality of electrode assemblies 220 into the two-dimensional partially collapsed configuration 308 of FIG. 3B. Also in the plurality of electrode assemblies 220 in the three-dimensional fully expanded configuration 314 of FIG. 3C, the first electrode assembly 230 in the first electrode assembly expanded configuration 306 can transition to the first electrode assembly collapsed configuration 302 and the second electrode assembly 240 in the second electrode expanded configuration 316 can transition to the second electrode collapsed configuration 304 to put the plurality of electrode assemblies 220 into the fully collapsed configuration 300 of FIG. 3A.

The electrode assemblies 220 are constructed in a manner to allow the first electrode assembly 230 to remain in the first electrode assembly expanded configuration 306 while the second electrode assembly 240 transitions between the second electrode assembly collapsed configuration 304 and the second electrode assembly expanded configuration 316. In one embodiment, the first spline assembly 232 is constructed from a shape memory metal alloy having a width and thickness that can remain in the first electrode expanded configuration 306 under the forces applied to the second spline assembly 242. For example, the first spline assembly 232 can remain in the first electrode assembly expanded configuration 306 until it is subjected to a force greater than a first deflection force. In some embodiments, the second spline assembly is constructed from a polymer or more pliable shape memory alloy that is able to transition under a second deflection force in which the second deflection force is less than the first deflection force. Thus, subjecting the plurality of electrode assemblies 220 to the second deflection force permits the second spline assembly 242 to transition between the second electrode assembly collapsed and expanded configurations 304, 316 without causing the first spline assembly 232 to transition.

In one embodiment, the first electrode assembly 230 (as well as the second electrode assembly 240) remains in the first electrode array collapsed configuration 302 while under a delivery sheath such that the plurality of electrode assemblies 220 are in the fully collapsed configuration. Once the sheath is retracted over the plurality of electrode assemblies 230, or the plurality of electrode assemblies 220 are extended from a distal end of the sheath, the shape set first spline assembly 232 transitions to the first electrode assembly expanded configuration 306. In one embodiment, the shape set second spline assembly 242 also transitions to the second electrode assembly expanded configuration 316. From the fully expanded configuration 314, a user can apply rotation or translation to the knob 214 on the handle 208 in a first direction to apply the second deflection force to the second electrode assembly 240 via the coupled transition member 212 to transition the second electrode assembly 240 to the second electrode assembly collapsed configuration 304 such as pulling the second electrode assembly 240 into the shaft lumen in the shaft distal regions 204 via the transition member 212. From the partially expanded configuration 308, a user can apply rotation or translation to the knob 214 on the handle 208 in a second direction opposite to the first direction to apply the second deflection force to the second electrode assembly 240 via the coupled transition member 212 to transition the second electrode assembly 240 to the second electrode assembly expanded configuration 316 such as pushing the second electrode assembly 240 out of the shaft lumen in the shaft distal region 204. The distal end of the delivery sheath can be extended over the first electrode assembly 230, or the first electrode assembly can be retracted into the delivery sheath, to transition the first electrode assembly 230 into the first electrode assembly collapsed configuration 302 (as well as the second electrode assembly if it is in the expanded configuration 316) to return the plurality of electrode assemblies 220 into the fully collapsed configuration 300.

In another embodiment, the first electrode assemblies 230 remains in the first electrode array collapsed configuration 302 while under a delivery sheath such that the plurality of electrode assemblies 220 are in the fully collapsed configuration. Once the sheath is retracted over the plurality of electrode assemblies 230, or the plurality of electrode assemblies 220 are extended from a distal end of the sheath, the shape set first spline assembly 232 transitions to the first electrode assembly expanded configuration 306. In one embodiment, the shape set second spline assembly 242 remains in the second electrode assembly collapsed configuration 304. From the partially expanded configuration 308, a user can apply rotation or translation to the knob 214 on the handle 208 in a first direction to apply the second deflection force to the second electrode assembly 240 via the coupled transition member 212 to transition the second electrode assembly 240 to the expanded configuration 316 such as via pushing the second electrode assembly 240 with the transition member 212 from the shaft distal region 204. From the fully expanded configuration 314, a user can apply rotation or translation to the knob 214 on the handle 208 in a second direction opposite to the first direction to apply the second deflection force to the second electrode assembly 240 via the coupled transition member 212 to transition the second electrode assembly 240 to the second electrode assembly collapsed configuration 304 such as via pulling the second electrode assembly 240 with the transition member 212 into the shaft distal region 204. The distal end of the delivery sheath can be extended over the first electrode assembly 230, or the first electrode assembly can be retracted into the delivery sheath, to transition the first electrode assembly 230 into the first electrode assembly collapsed configuration 302 (as well as the second electrode assembly if it is in the second electrode assembly expanded configuration 316) to return the plurality of electrode assemblies 220 into the fully collapsed configuration 300.

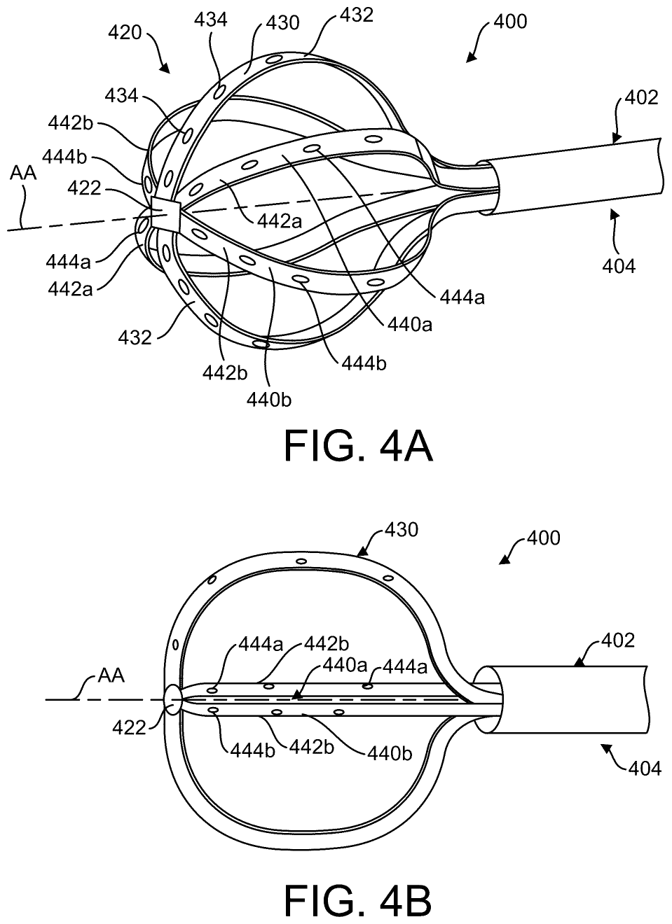

FIGS. 4A and 4B illustrate another embodiment of a catheter 400 configured to detect a plurality of physiological signals from within a patient's heart. The catheter 400 corresponds with the catheter 105 described with respect to FIG. 1 and can be used in the electrophysiology system 50. For example, catheter 400 can be configured as a mapping catheter in some embodiments and as a mapping and ablation catheter via irreversible electroporation in some embodiments. The catheter 400 has an elongated shaft 402 having a proximal region and a shaft distal region 404 configured to be deployed proximate target tissue, such as within a chamber of the patient's heart. The elongated shaft 402 defines a longitudinal axis AA, which is presented as a line passing through a centroid of a cross section of the shaft 402. In the illustrated embodiment, the catheter 402 includes a location sensor (not shown) coupled to the distal region 404, such as within the shaft 402, in which the location sensor can be configured to generate a plurality of location signals representative of a location of the catheter 400 within the patient's heart. A plurality of electrode assemblies 420 extend distally from the distal region 404 of the shaft 404. The plurality of electrode assemblies 420 are coupled together at a distal hub 422.

Catheter 400 illustrates an embodiment of catheter 200 of FIG. 2 having a plurality of second electrode assemblies. In the illustrated embodiment, the plurality of electrode assemblies 420 include a first electrode assembly 430 and a plurality of second electrode assemblies 440a, 440b. The first electrode assembly 430 includes a first spline assembly 432. A first plurality of electrodes 434 are disposed on the first spline assembly 432. The second electrode assemblies 440 include second spline assemblies 442a, 442b coupled to the first spline assembly 432 at the distal hub 422. A second plurality of electrodes 444a, 444b are disposed on the second spline assemblies 442a, 442b respectively, the first and second electrode assemblies 430, 440a, 440b can be constructed in a manner similar to electrode assemblies 230, 240 of catheter 200.

FIG. 4A illustrates the plurality of electrode assemblies 420 of catheter 400 in a fully expanded configuration. In particular, the first electrode assembly 430 is in the first electrode assembly expanded configuration and the second electrode assemblies 440a, 440b are each in a second electrode assembly expanded configuration. The first electrode assembly 430 in the first electrode assembly expanded configuration is defined as the first planar loop, and each of the second electrode assemblies 440a, 440b in the second electrode assembly expanded configuration are defined as a plurality of second loops out of the plane of the first electrode assembly 430. Accordingly, the fully expanded configuration defines a three-dimensional basket shape. In the second electrode assembly expanded configuration, the second spline assemblies 442a, 442b are extended from the shaft lumen in the shaft distal region 404. Electrodes configured for sensing or the sensing electrodes disposed on the plurality of electrode assemblies 420 are exposed and distal to the shaft 402. Also, electrodes configured for ablation or the ablation electrodes disposed on the plurality of electrode assemblies 420 are exposed and distal to the shaft 402.

FIG. 4B illustrates the plurality of electrode assemblies 420 of the catheter 400 each in a partially expanded configuration or a partially collapsed configuration. In particular, the first electrode assembly 430 is in a first electrode assembly expanded configuration and the second electrode assemblies 440a, 440b are each in a second electrode assembly collapsed configuration. The first electrode assembly 430 in the first electrode assembly expanded configuration is defined as a first planar loop, and the second electrode assembly 440 in the second electrode assembly collapsed configuration are defined as a longitudinally extending cylinder in the plane of the first electrode assembly 430. For instance, the second spline assemblies 442a, 442b are each in plane with the first spline assembly 432. The proximal ends of the second spline assemblies 442a, 442b are disposed within the lumen of the shaft 402 such that the second spline assemblies 442a, 442b is not fully extended from the shaft 402. Accordingly, the partially expanded or partially collapsed configuration defines a two-dimensional paddle shape configured in the plane. Electrodes configured for sensing or the sensing electrodes disposed on the plurality of electrode assemblies are exposed and distal to the shaft 402. In some embodiments, some or all electrodes configured for ablation or ablation electrodes are disposed within the shaft lumen in the shaft distal region 404. In some examples, one of second electrode assemblies can assume the collapsed configuration and the other of the second electrode assemblies 420 can assume the expanded configuration.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter to detect a plurality of physiological signals from within a patient's heart, the catheter comprising:
an elongated shaft having a proximal region and a distal region, the elongated shaft defining a longitudinal axis; and
a plurality of electrode assemblies extending distally from the distal region of the shaft and coupled together at a distal hub, the plurality of electrode assemblies including a first electrode assembly and a second electrode assembly;
the first electrode assembly transitionable between a first electrode assembly expanded configuration and a first electrode assembly collapsed configuration, the first electrode assembly comprising:
a first spline assembly configured in a first planar loop in the first electrode assembly expanded configuration, and
a first plurality of electrodes disposed on the first spline assembly;
the second electrode assembly transitionable between a second electrode assembly expanded configuration and a second electrode assembly collapsed configuration when the first electrode assembly is in the first electrode assembly expanded configuration, the second electrode assembly comprising:
a second spline assembly coupled to the first spline assembly at the distal hub, the second spline assembly configured in a second loop out of plane from the first planar loop in the second electrode assembly expanded configuration and in plane with the first planar loop in the second electrode assembly collapsed configuration, and
a second plurality of electrodes disposed on the second spline assembly.

2. The catheter of claim 1, wherein the first electrode assembly in the first electrode assembly expanded configuration and the second electrode assembly in the second electrode assembly expanded configuration are transitionable to the first electrode assembly collapsed configuration and the second electrode assembly collapsed configuration.

3. The catheter of claim 1, wherein the first electrode assembly in the first electrode assembly collapsed configuration and the second electrode assembly in the second electrode assembly collapsed configuration are transitionable to the first electrode assembly expanded configuration and the second electrode assembly expanded configuration.

4. The catheter of claim 1, wherein the plurality of electrodes includes a set of sensing electrodes and a set of ablation electrodes.

5. The catheter of claim 1, wherein the first spline assembly in the first electrode assembly expanded configuration includes a first curved spline having a first distal end and an opposite, second curved spline having a second distal end, the first and second distal ends coupled to the distal hub.

6. The catheter of claim 1, wherein the first spline assembly is configured from a nickel-titanium alloy.

7. The catheter of claim 1, wherein the elongate shaft includes a transition member coupled to the second electrode assembly to transition the second electrode assembly between the second electrode assembly expanded configuration and the second electrode assembly collapsed configuration.

8. The catheter of claim 7, and further comprising a handle having a knob, wherein the knob is coupled to the transition member.

9. The catheter of claim 8, wherein the knob is configured to rotate about the longitudinal axis, and rotation of the knob transitions the second electrode assembly between the second electrode assembly expanded configuration and the second electrode assembly collapsed configuration.

10. The catheter of claim 8, wherein the knob is configured to translate along the longitudinal axis, and translation of the knob transitions the second electrode assembly between the second electrode assembly expanded configuration and the second electrode assembly collapsed configuration.

11. The catheter of claim 1, wherein the second spline assembly includes a proximal end, and the proximal end is configured to be disposed within the elongated shaft in the second electrode assembly collapsed configuration and to slide out of the elongated shaft as the second electrode assembly transitions from the second electrode assembly collapsed configuration to the second electrode assembly expanded configuration.

12. The catheter of claim 1, wherein the plurality of electrode assemblies includes one first electrode assembly and one second electrode assembly.

13. The catheter of claim 1, wherein the plurality of electrode assemblies include a first electrode assembly and plurality of second electrode assemblies.

14. The catheter of claim 13, wherein the second electrode assemblies are each transitionable between a second electrode assembly expanded configuration and a second electrode assembly collapsed configuration when the first electrode assembly is in the first electrode assembly expanded configuration.

15. The catheter of claim 13, wherein the plurality of electrode assemblies include one first electrode assembly and two second electrode assemblies.

16. A catheter to detect a plurality of physiological signals from within a patient's heart, the catheter comprising:
an elongated shaft having a proximal region and a distal region, the elongated shaft defining a longitudinal axis; and
a plurality of electrode assemblies extending distally from the distal region of the shaft and coupled together at a distal hub, the plurality of electrode assemblies including a first electrode assembly and a second electrode assembly, wherein the plurality of electrodes includes a set of sensing electrodes and a set of ablation electrodes;
the first electrode assembly transitionable between a first electrode assembly expanded configuration and a first electrode assembly collapsed configuration, the first electrode assembly comprising:
a first spline assembly configured in a first planar loop in the first electrode assembly expanded configuration, and
a first plurality of electrodes disposed on the first spline assembly;
the second electrode assembly transitionable between a second electrode assembly expanded configuration and a second electrode assembly collapsed configuration when the first electrode assembly is in the first electrode assembly expanded configuration, the second electrode assembly comprising:
a second spline assembly coupled to the first spline assembly at the distal hub, the second spline assembly configured in a second loop out of plane from the first planar loop in the second electrode assembly expanded configuration and in plane with the first planar loop in the second electrode assembly collapsed configuration, and
a second plurality of electrodes disposed on the second spline assembly.

17. The catheter of claim 16, wherein the first electrode assembly in the first electrode assembly expanded configuration and the second electrode assembly in the second electrode assembly expanded configuration are transitionable to the first electrode assembly collapsed configuration and the second electrode assembly collapsed configuration.

18. The catheter of claim 17, wherein the first electrode assembly in the first electrode assembly collapsed configuration and the second electrode assembly in the second electrode assembly collapsed configuration are transitionable to the first electrode assembly expanded configuration and the second electrode assembly expanded configuration.

19. A system to detect a plurality of physiological signals from within a patient's heart, the system comprising:
an electroporation console configured to deliver electric field energy to targeted tissue in the patient's heart, the electroporation console coupled to a catheter, the catheter comprising:
an elongated shaft having a proximal region and a distal region, the elongated shaft defining a longitudinal axis; and
a plurality of electrode assemblies extending distally from the distal region of the shaft and coupled together at a distal hub, the plurality of electrode assemblies including a first electrode assembly and a second electrode assembly, wherein the plurality of electrodes includes a set of sensing electrodes and a set of ablation electrodes, the electric field energy delivered to the set of ablation electrodes;
the first electrode assembly transitionable between a first electrode assembly expanded configuration and a first electrode assembly collapsed configuration, the first electrode assembly comprising:
a first spline assembly configured in a first planar loop in the first electrode assembly expanded configuration, and
a first plurality of electrodes disposed on the first spline assembly;
the second electrode assembly transitionable between a second electrode assembly expanded configuration and a second electrode assembly collapsed configuration when the first electrode assembly is in the first electrode assembly expanded configuration, the second electrode assembly comprising:
a second spline assembly coupled to the first spline assembly at the distal hub, the second spline assembly configured in a second loop out of plane from the first planar loop in the second electrode assembly expanded configuration and in plane with the first planar loop in the second electrode assembly collapsed configuration, and
a second plurality of electrodes disposed on the second spline assembly.

20. The system of claim 19, and further comprising an electroanatomical mapping system coupled to the set of sensing electrodes.
